(19) United States
(12) Patent Application Publication
(10) Pub. No.: US 2021/0244904 A1
(43) Pub. Date: Aug. 12, 2021

(54) TUBE FASTENER DEVICE

(71) Applicant: The Court of Edinburgh Napier University, Edinburgh (GB)

(72) Inventors: Cameron James McEwan Perrie, Edinburgh (GB); Elizabeth Christina Adamson, Edinburgh (GB); William Ernest Titley, Edinburgh (GB)

(21) Appl. No.: 16/763,669

(22) PCT Filed: Nov. 13, 2018
(86) PCT No.: PCT/GB2018/053285

§ 371 Date: May 13, 2020

(30) Foreign Application Priority Data
Nov. 13, 2017 (GB) 1718747.7

(51) Int. Cl. A61M 16/04; A61M 16/08

(52) U.S. Cl. CPC A61M 16/0497; A61M 16/0875; A61M 2205/02; A61M 2209/06; A61M 2209/088

(57) ABSTRACT

There is described a tube fastener, in particular tube fastener to secure respiratory tubing for breathing systems, methods of using the tube fastener, a carriage for use with the tube fastener and a system.

### FIELD OF THE INVENTION

This invention relates broadly to the field of tube fasteners and is particularly concerned with the securing of medical tubing with respect to a patient, suitably respiratory tubing for breathing systems.

### BACKGROUND TO THE INVENTION

A key part of intensive medical care is the controlled delivery or removal of fluids, either gases or liquids to or from patients. In either case, the patient is connected to a dispensing or collecting means via hollow tubing or tubes which may be delicately attached at sensitive sites to the patient to permit fluid transference. For example, endotracheal tubing or tubes may be secured to a patient's neck to permit ventilation. In another procedure medical tubing or tubes may be attached to a patient's arm to permit intravenous medicament delivery. In all cases a sudden pull on the hollow tubing may cause medical complications; as such, it is necessary to secure the tubing and minimise pulling motions.

In the case of a tracheostomy: a stoma is created in the trachea to provide air to the lungs while circumventing the mouth via a hole in the neck. For the stoma to remain open and functional (i.e. permit gas transference), a tracheostomy tube is provided to the stoma. While some patients are able to breathe through the tracheostomy tube, others require the use of ventilators, which must be connected to the tracheostomy tube using hollow tubes.

Other means of gas or liquid delivery or removal include extracranial shunts, male or female urinary catheters, arterial haemodynamic monitoring and aortic or portal blood sampling. In all cases, patients are connected to reservoirs or collectors using hollow tubing/tubes, where the hollow tubing is usually connected at sensitive sites. As stated, it is essential that once in place, the hollow tubing is securely fastened in position, and not for example accidentally pulled away: to do so could cause problems and complications for the patient.

In addition to the delivery or removal of liquids and gases to patients, intensive care patients are frequently monitored for critical information such as breathing rate, heart rate and blood pressure. This information is gathered by sensors attached to the patient (typically via adhesive pads) and connected to monitoring devices by hollow tubing containing electrical cabling.

In all of the above cases, the patient is connected at sensitive sites to devices via hollow tubing. Frequently a number of hollow tubes are provided to the patient, which can become entangled and confusing to medical staff at critical moments. For reasons of patient safety, several methods have been proposed to organise the hollow tubing, such as GB 2470483 and U.S. Pat. No. 5,316,246.

Through advances in medical science, greater numbers of people including children are able to be achieve mobility and the associated enhanced quality of life, despite the requirement for a medical device such as home ventilation. For all mobile patients the accidental pulling of tubes can be a particular concern.

### SUMMARY OF THE INVENTION

The present invention provides a device to secure medical tubes to inhibit the tubes being accidentally pulled away from a patient.

Accordingly, a first aspect of the present invention provides a device for holding a flexible tube, the device comprising a body member, the body member comprising at least one tube receiving portion, the at least one tube receiving portion comprising one or more channels adapted to accommodate a portion of a flexible tube with a first and second end to be held, wherein;
a channel provides a surface which, in use, resists longitudinal movement of the tube in the channel for example by providing a friction fit to the tube, in use located within the channel, when the tube is provided in the tube receiving portion, such that;
when the tube is pulled at one end away from the device, the device inhibits the movement of the tube and the pulling force is substantially reduced at the second end.

Suitably the device may comprise a plurality of channels wherein the channels are adapted to accommodate a portion of tubing in one or more channels for example by a friction fit. Suitably, a friction fit may be provided by providing a channel with dimensions (cross-section) slightly less than the tube or tubing to be accommodated therein such that when a portion of the tube or tubing is provided by press fitting in the channel the tube is held in the channel. Suitably the tube may be located in the tube and held via a clamping action to prevent the motion of the tubing. Suitably, the tube may be held by a transition fit such that the tube is inserted into the channel by hand with a light force. Suitably further protrusions or flanges are used to interlock the tube to the body member. Suitably, the friction fit may be provided by a snap fit, for example by deflection of a channel surface when the tube is inserted therein.

Suitably, the device may comprise one or more channels wherein the channels further comprise protrusions extending from the channel surface into the channel to inhibit the movement of tubing upon pulling. Suitably the protrusions may be friction inducing. Suitably one or more protrusions may be one or more flanges located on a surface of or at an end(s) of a channel.

Suitably the tube may be a ventilator tube or a tube which has an external surface which comprises tubing protrusions, optionally wherein the tubing protrusions comprise one or more helices or ribs to form raised portions and recesses on the exterior of the tube or a portion thereof. The channel protrusions may be arranged to interlock with the recesses provided on the tube between the protrusions on the exterior of the tube.

Suitably, the one or more channels extend in length from a first surface of the body member to a second opposing surface of the body member. Suitably the one or more channels may be fully enclosed. Alternatively, the one or more channels may have an opening in the cross sectional perimeter of the channel along the length of the channel of at least 10% of the perimeter, at least 20% or the perimeter, at least 30% of the perimeter, at least 40% of the perimeter, at least 50% of the perimeter.

The friction fit may be enhanced by providing flanged portions to interlock with external portions provided on the tubing. As will be appreciated the body member or a portion thereof may be formed from resilient material which allows a tube to be pushed into a channel, but a sufficient friction fit to be achieved. As will be appreciated where protrusions or flanges are provided at a channel surface or at an end(s) of the channel, the friction fit of the tube to the channel can be less tight as the protrusions or flange(s) will inhibit longitudinal movement of the tube.

Suitably, the at least one tube receiving portion may be composed of hard plastic, deformable plastic, rubberised plastic or a mixture thereof.

Suitably the device may have one or more channels to fit the tubing through scaling and shaping of the one or more channels. Suitably the channels may be provided with inserts to alter the size of the channel to accommodate a portion of tubing in the one or more channels to permit a variety of tubing diameters to be provided. The insert may be removable. The advantage of this is that the device may be suitable to secure a number of diameters of medical tubing, for a variety of uses by application of an insert to reduce the cross sectional area and perimeter of the channel. This is particularly advantageous in the case of securing ventilator tubing; commonly this is provided in diameters of 10 mm, 15 mm, 22 mm or 30 mm, which may be used to treat neonatal, paediatric, and adult patient groups respectively.

Suitably a tube with at least first and second ends is provided to the device such that a portion between the ends of the tube rests in the one or more channels of the device such that if one end of the tube is pulled away from the device, the device inhibits the movement of the tubing and the pulling force is substantially reduced at the second end. In embodiments of the device, the transmitted pulling force is reduced by more than 100 N, up to 500 N, up to 1 kN, up to S kN, up to 10 kN up to 15 kN or more than 15 kN. Suitably the friction fit or interlocking can inhibit pulling forces of at least 5 kN, at least 10 kN at least 15 kN or at least 15 kN.

Advantageously, one or more flanges or one or more protrusions may interlock with ribs provided on an exterior of the ventilator tube to provide an added level of friction or impedance to removal of a tube located in a channel, particularly where a flange is arranged to interlock with a feature on the exterior of a tube, for example a rib on a flexible tube for a ventilator tube.

Suitably the one or more channels of the device extend from a first surface of the body member to a second opposing surface of the body member.

Suitably when a tube is located in a channel of the device of the first aspect of the invention, the channel(s) may be fully enclosed, by a tube locating member. Suitably a tube locating member may releasably attach over a channel such that when a tube is located in the channel the tube locating member can be provided over a first portion of the tube to be held within the channel. Suitably the tube locating member may be a removable member which may be releasably fixed to the body member. The reversible fixation may be provided by any suitable means, for example the locating member may comprise a longitudinal member with flanged portions at first and second ends of the longitudinal member and a corresponding body member may comprise a first and second recess to receive a corresponding flanged portion of the locating member such that the locating member can be provided over the at least one channel and be held over the channel to resist movement of a tube located in the channel by the engagement of the flanged portions in the respective recess of the body member.

Alternatively a locating member may be provided by a belt located over the channels and secured in position by a hook and loop fastener, for example Velcro™. Alternatively the device may be provided in a housing, the housing acting as a locating member that locates and retains a tube(s) in a channel of the device.

Suitably the channels may be partially enclosed. Suitably the device may comprise both fully or partially enclosed channels. This would provide either holes or grooves adapted to the tubing, where the tubing may be laced through the holes or slotted into the grooves. The amount to which the one or more channels are partially enclosed may affect the friction fit of the tubing, where a partially enclosed channel of say 60% enclosed provides a smaller surface area to grip the outer surface of the channel, providing a smaller reduction in transmitted pulling force compared with a partially enclosed channel of say 80%, which comparatively provides an increased surface area. Similarly, increasing the amount of partial channel enclosure increases the force required to slot the tubing into the channel.

In some embodiments the at least one tube receiving portion of the body member can be composed of hard plastic, in some embodiments it can be composed of deformable plastic, in some embodiments it can be rubberised. These options allow the device to be suitable for a number of uses; hard plastic may provide increased durability, deformable plastic may mean that slotting the tubing into partially enclosed channels requires a smaller force than if the at least one tube receiving portion was composed of hard plastic. A rubberised tube receiving portion may permit a greater frictional force between the surface of the channel and the tubing decreasing the transmitted pulling force.

Suitably the device may be attached to a support by a connector. This permits the transference of pulling force from one end of the tube/tubing to the support, and prevents the transmittance of pulling force to the second end of the tube/tubing. Suitably the support member may be a belt, strap, stud, clip or clamp. Suitably the connector on the device may be a belt groove provided in a surface arranged to receive a belt or strap. Suitably the support may be a vest worn by a patient and the device may be attached to the vest, by for example a belt or strap or other suitable means. Suitably the device may be shaped to conform to the part of the body to which the device is to be attached, i.e. curved or arcuate to conform to the small of the back or flat to conform to the front of the chest of a patient.

Advantageously, the body member may be provided in a body member housing arranged to at least partially enclose the channel(s), this prevents the accidental removal of the tube from the one or more channels by translational force.

Suitably at least three channels may be provided in the body member to form a tube receiving portion. This increases the reduction in pulling force translation from the first end of the tube/tubing to the second end as it allows a greater proportion of the tube to be provided in a channel(s). Suitably at least three channels may be arranged such that a first and second channel are arranged substantially parallel to each other, with a third channel interposed between the first and second channels (also arranged in parallel to the first and second channels). This arrangement of channels permits a sigmoidal configuration of tubing to be arranged between the three channels, increasing the resistance of pulling force while minimising crushing of the tubing. Providing the tubing to the one or more channels such that a portion of tubing between the first and second end is between the first, second and third channels may increase the reduction of transmitted pulling force due to constrictive gripping of the tube by the device.

According to a second aspect of the invention there is provided a kit comprising the device of the first aspect and a tube, suitably a ventilator tube. In embodiments the kit may comprise a belt to attach the device to a patient.

According to a third aspect of the device there is provided a mobile ventilator system comprising a carriage for a ventilator or gas or fluid, a ventilator tube and the device of the first aspect of the invention. Suitably the carriage may be able to be tethered to a patient to allow them to pull the carriage. The carriage may be tethered to the patient via a belt or harness worn by the patient. For example, D rings may be provided to a belt or harness worn by the patient such that the carriage may be connected to the belt or harness. Suitably the device may be attached to the belt or harness. Suitably the carriage may be attached to a belt, harness or vest worn by the user via a pole or poles. Suitably the carriage may be attached to a belt, harness or vest via telescopic poles, for example telescopic aluminium poles.

In use, such a system allows a patient requiring use of the ventilator increased mobility as the ventilator can be located in a carriage and pulled by the patient. The device allows improved tube management and minimises the risk of a tube extending from a ventilator to a patient being accidentally pulled from the patient or ventilator by inhibiting movement of the tube along its longitudinal axis. Suitably the device may also inhibit movement of the tube relative to the patient.

According to a fourth aspect of the invention there is provided a carriage for use with a device of the first aspect of the invention.

Suitably the carriage may comprise two or more wheels. In embodiments comprising two wheels, these may be arranged at around the mid portion of a first and opposite side of the carriage.

Suitably the carriage may be formed from water-jet cut foam. Suitably a carriage formed from water jet cut foam may have a base formed from metal, for example aluminium, or a plastics material. Suitably the carriage may be provided with a cover which in use is provided over the material being transported by the carriage, for example a ventilator, gas, or fluid being carried by the carriage. Suitably the cover may be a rain cover, for example formed from water resistant material or fabric. Suitably, the carriage may be formed by 3D printing, injection moulding or any other suitable means in the art.

According to a fifth aspect of the invention there is provided the use of the device of the first aspect of the invention, the kit of the second aspect of the invention, the system of the third aspect of the invention or the carriage of the fourth aspect of the invention.

Embodiments of the invention will now be provided by way of example only with reference to the accompanying figures in which:

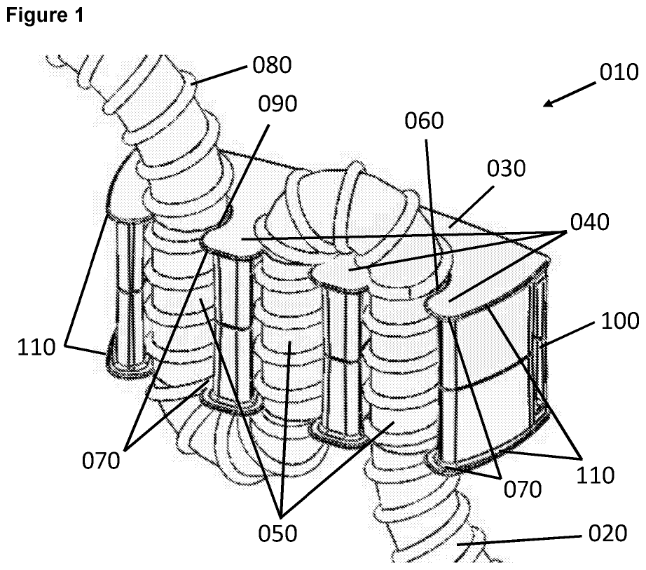

FIG. 1 shows an illustration of an embodiment of the device of the invention.

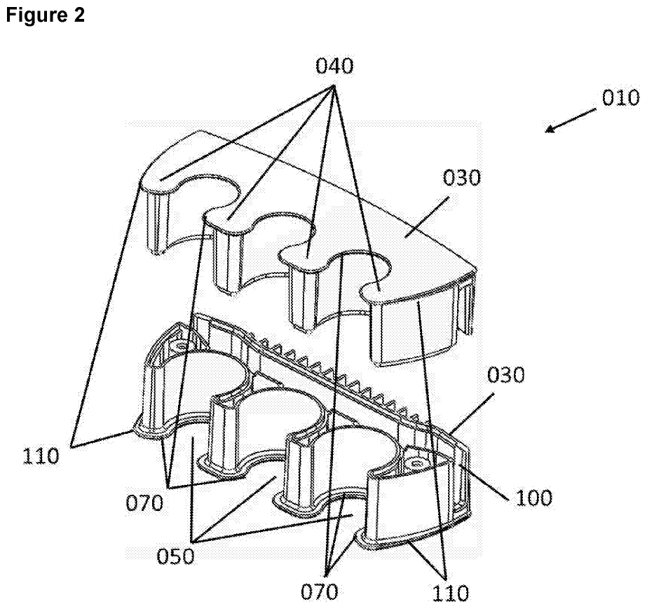

FIG. 2 shows an illustration of an injection moulded embodiment of the invention, wherein the body of the device is provided as two portions which are conjoined together.

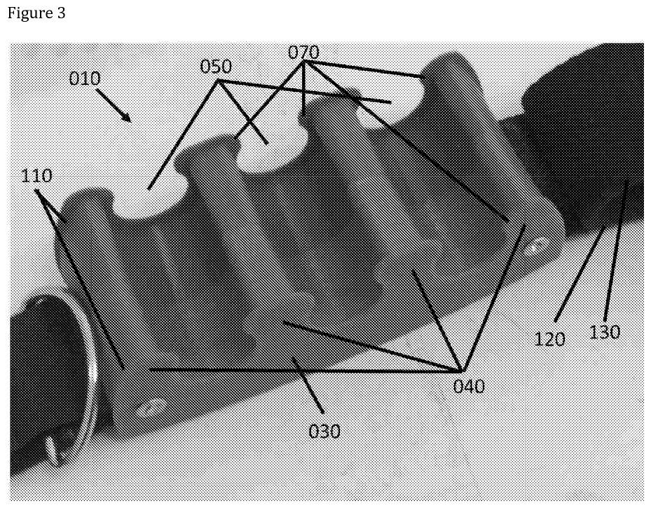

FIG. 3 shows a device of the present invention provided on a belt support member, wherein the belt support member is provided in a recess of the device.

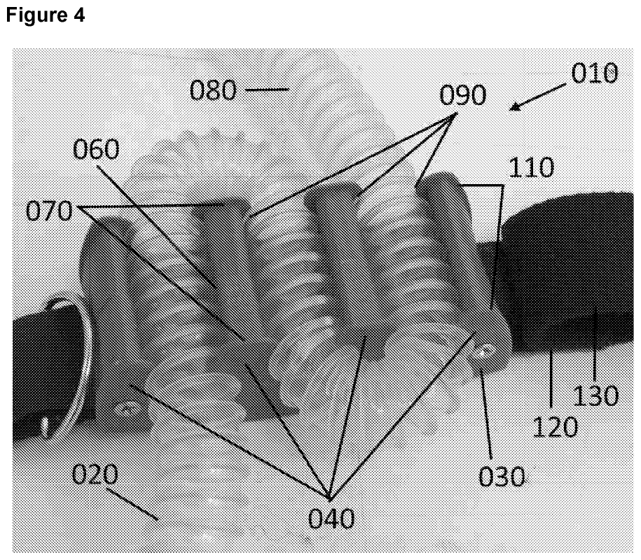

FIG. 4 shows a tube located in the channels of an embodiment of the device wherein flanges at ends of the channels interlock with ribs/recesses provided on the exterior of the tube to inhibit movement of the tube along its longitudinal axis and wherein the tube is provided in a sigmoidal arrangement in the channels of the device.

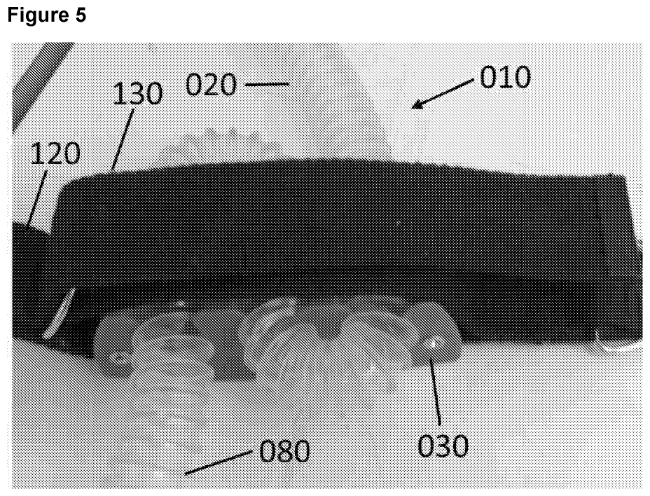

FIG. 5 shows a belt locating member provided over the channels of the device member to further inhibit the removal of the tube located within the channels of the device.

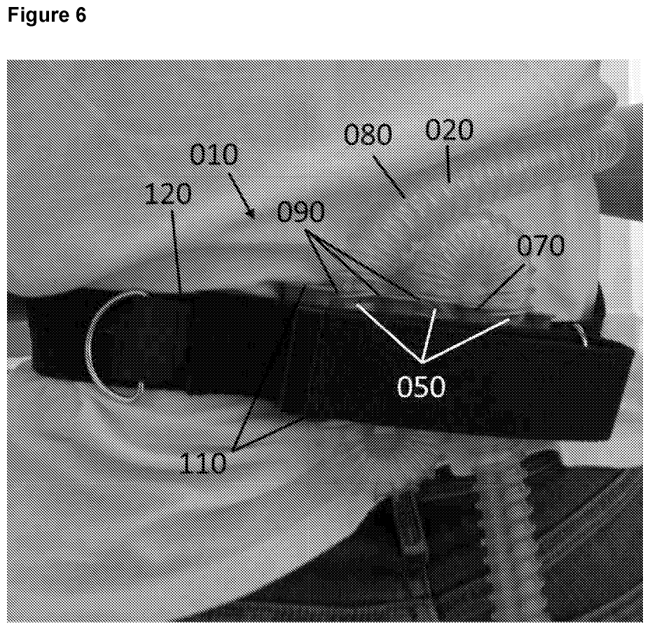

FIG. 6 shows an embodiment of the device in use, with a ventilator tube located in the channels of the device and inhibited from movement along the longitudinal axis of the tube by both the sigmoidal arrangement of the tube located in the channels of the device and by the flange portions located at the first and second ends of each of the channels; also shown is a tube locating member being provided by a belt portion releasably attached over the channels using hook and loop fasteners.

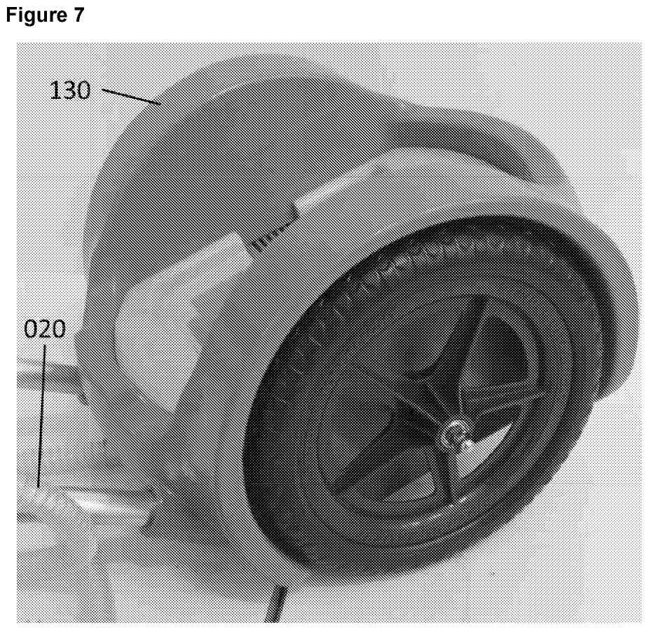

FIG. 7 shows a carriage suitable for use with a device of the present invention.

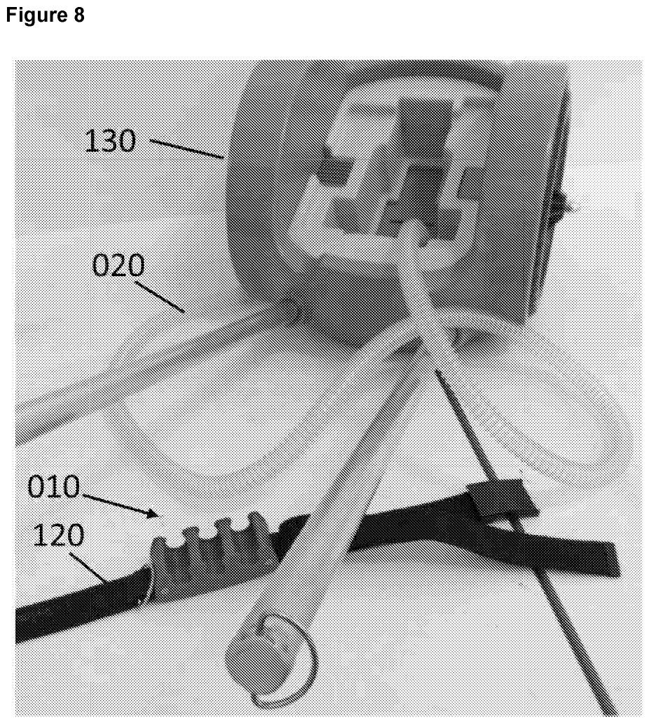

FIG. 8 shows a system of the present invention, with a carriage to hold a respirator, a ventilator tube to allow transfer of gas from the ventilator to a patient, a device of the present invention and a belt support member to allow the device to be located to a patient.

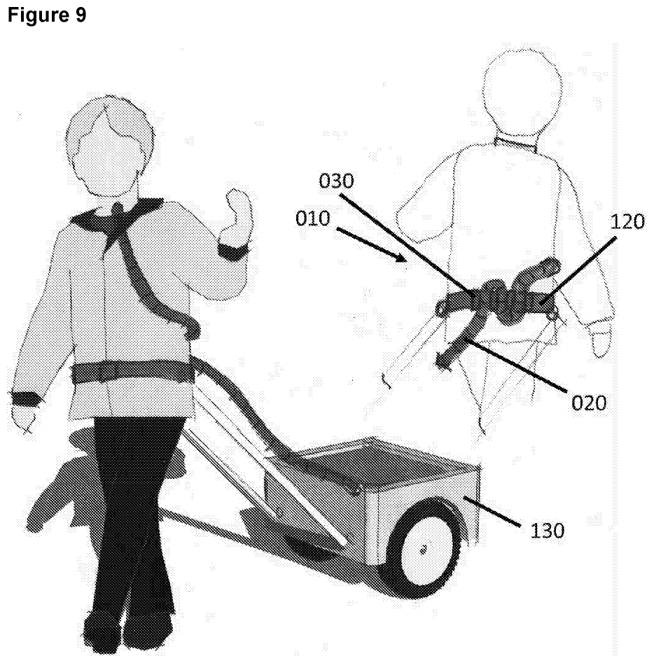

FIG. 9 shows an embodiment of the system of the present invention in use.

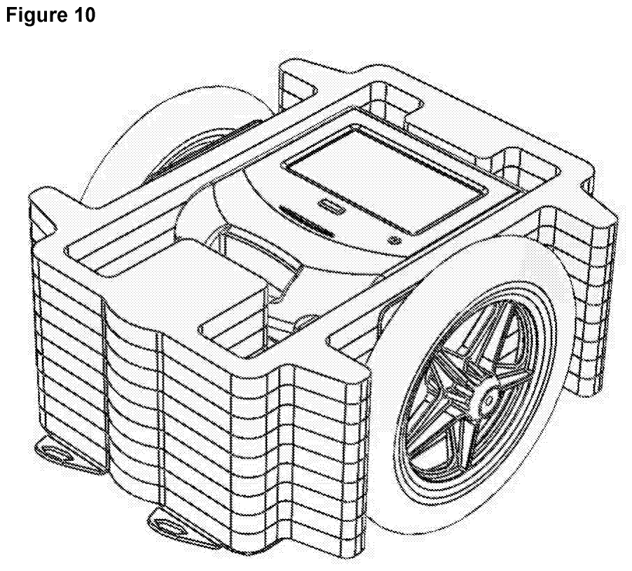

FIG. 10 shows an embodiment of a carriage to hold a respirator.

### DEFINITIONS

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

As used herein, the articles “a” and “an” refer to one or to more than one (for example to at least one) of the grammatical object of the article.

“About” shall generally mean an acceptable degree of error for the quantity measured given the nature or precision of the measurements.

### DETAILED DESCRIPTION OF THE INVENTION

An embodiment of the invention is shown as an example in FIG. 1, where the device 010 is shown securing a portion of medical tubing 020. The device 010 comprises a body portion 030 comprised of at least one tube receiving portion 040 comprising one or more channels 050 (in this example three partially enclosed channels where the first and second channel are substantially parallel and the third channel is interposed between them) adapted to accommodate a portion of the tube to be held, where the surface provides a friction fit 060 to a tube when the tube is provided to the tube receiving portion of the device. In this embodiment the channels 050 further comprise friction inducing protrusions 070, in this case two flanges arranged at each end of the channels. In this embodiment the tube to be held 020 is a ventilator tube whose external surface comprises protrusions 080, in this case formed by a single helix. In this example the channel protrusions 070 are arranged to interlock 090 with the tubing protrusions 080. In this embodiment the body member further comprises a connector 100 adapted to attach the body member to a support member 120, in this embodiment the connector is a belt groove adapted to receive a belt or strap. In this embodiment the body member is arranged 110 to receive a cover member to enclose the partially enclosed channels. In this embodiment the tube 020 is provided through three channels in a sigmoidal configuration, with no excess tubing provided between channels.

An embodiment of the system of the invention is shown as an example in FIG. 9 wherein the device 010 is shown securing a portion of medical tubing 020 to a support member 120. In this embodiment the system comprises a mobile ventilator system comprising a carriage for a ventilator and a ventilator 130, connected to the support member 120 by bars (for example telescopic poles) and D rings. Suitably a carriage may be formed by any suitable method known in the art, for example water jet cut foam, injection moulding or the like. Suitable the carriage may be provided with wheels, for example 2 or 4 wheels provided at the side of the carriage to allow the carriage to move easily.

A method of manufacturing the device 010 is plastic injection moulding of the body member 030, where the device is produced as two halves affixed together. An example of this can be seen in FIG. 2 where no tubing 020 has been provided. This diagram provides a greater view of the one or more channels 050, the one or more channel protrusions 070 in this case flanges, and the connector 100 adapted to attach the body member to a support member, in this embodiment the connector is a belt groove adapted to receive a belt or strap.

An embodiment of the carriage and system of the invention is shown as an example in FIG. 10 wherein the carriage, formed of water jet cut foam with a metal base, is shown with a recess adapted to receive a ventilator. Forming the carriage using water jet cut foam is advantageous as it allows rapid and cheap production of the carriage. In this embodiment the carriage comprises a wheel located on the carriage or either side of the mobile ventilator system. As illustrated, projections from either side of the carriage can be provided to protect the patient or others being brought into contact with the wheels. The carriage can be connected to the patient via telescopic poles which can be attached to a belt, vest or harness worn by the user.

Although the invention has been particularly shown and described with reference to particular examples, it will be understood by those skilled in the art that various changes in the form and details may be made therein without departing from the scope of the present invention.

1. A device for holding a flexible tube with a first and second end, the device comprising a body member, the body member comprising at least one tube receiving portion, the at least one tube receiving portion comprising one or more channels adapted to accommodate a portion of a flexible tube to be held, wherein;
a channel provides a surface which provides a friction fit to the tube, in use located within the channel, when the tube is provided in the tube receiving portion, such that;
when the tube is pulled at a first end away from the device, the device inhibits the movement of the tube and the pulling force is substantially reduced at the second end.

2. The device of claim 1 wherein the at least one tube receiving portion is composed of hard plastic, deformable plastic, rubberised plastic or a mixture thereof.

3. The device of claim 1 or 2 wherein the tube receiving portion comprises at least three channels.

4. The device of any of the preceding claims wherein the one or more channels further comprise protrusions extending from the channel surface into the channel to inhibit the movement of the tube.

5. The device of claim 4 wherein the one or more protrusions comprise one or more flanges located at an end(s) of a channel.

6. The device of any of the preceding claims wherein the tube is a ventilator tube, wherein the ventilator tube has an external surface which comprises tube protrusions, optionally wherein the tube protrusions comprise one or more helices or ribs.

7. The device of claim 6 wherein the channel protrusions are arranged to interlock with the recesses provided on the tube between the protrusions on the exterior of the tube.

8. The device of any of the preceding claims wherein the one or more channels extend in length from a first surface of the body member to a second opposing surface of the body member.

9. The device of any of the preceding claims wherein at least one of the one or more channels are fully enclosed.

10. The device of any one of claims 1 to 8 wherein at least one of the one or more channels have an opening in the cross sectional perimeter of the channel along the length of the channel of at least 30% of the perimeter.

11. The device of any of the preceding claims wherein the body member further comprises a connector to attach the body member to a support member.

12. The device of claim 11 wherein the support member comprises a belt, strap, clip or clamp.

13. The device of claim 11 or 12 wherein the support member comprises a belt and the connector is a groove on a surface of the body member, the groove configured to receive the belt.

14. The device of any of the preceding claims further comprising a housing wherein the housing is arranged to receive the body member to enclose the channels.

15. The device of any preceding claim comprising at least three channels wherein a first and second channel are arranged substantially parallel to each other with a third channel interposed between the first and second channel.

16. The device of any preceding claim wherein the device comprises three channels, wherein the three channels are provided in a first surface of the body member, each channel comprising a first flange at a first end of the channel and a second flange at a second opposite end of the channel,
wherein the channels are adapted to accommodate a ventilator tube and in use the flanges are arranged to interlock with recesses provided on an exterior of the ventilator tubes located in the channel.

17. Use of the device of any of the preceding claims wherein;
a tube with first and second ends is provided to the device such that a portion of the tube between the ends is located in a channel, and wherein;
should the first end of the tube be pulled away from the device, the device inhibits the movement of the tube along the longitudinal axis of the tube.

18. Use of a device of claim 17 wherein;
a tube with first and second ends is provided to the device such that a portion of the tube between the ends is located in a first, second and third channel.

19. Use as claimed in claim 18 wherein the portion of the tube is provided through the three channels in a sigmoidal configuration.

20. Use as claimed in claim 18 or 19 wherein the tube extends between the one or more channels using a minimum length of tube with respect to the distance between the channels.

21. A kit comprising flexible tubing and the device of any of claims 1 to 16.

22. A mobile ventilator system comprising a carriage for a ventilator or gas or fluid, a ventilator tube and the device of any of claims 1 to 16.

23. A carriage for use with a device of any one of claims 1 to 16.
